(19) United States
(12) Patent Application Publication
(10) Pub. No.: US 2023/0067029 A1
(43) Pub. Date: Mar. 2, 2023

(54) Arylalkylamine, pyrrole, indole, and opiate derivative concentration determination method and test kit using said method

(71) Applicant: Felix Blei, Jena (DE)

(72) Inventor: Felix Blei, Jena (DE)

(21) Appl. No.: 17/624,476

(22) PCT Filed: Jun. 30, 2020
(86) PCT No.: PCT/DE2020/100566

§ 371 Date: Nov. 14, 2022

(30) Foreign Application Priority Data
Jul. 1, 2019 (DE) 10 2019 117 739.1

(51) Int. Cl. G01N 33/94; G01N 31/22

(52) U.S. Cl. CPC G01N 33/946; G01N 31/22

(57) ABSTRACT

The invention relates to an arylalkylamine, pyrrole, indole and opiate derivative concentration determination method and a test kit using said method for precise determination of the presence and precise concentration of said substances which have an indole as a structural element and similarly structured substances having primary amines or pyrrole compounds, phenylethylamines and other substances. The problem addressed by the invention of is that of providing an arylalkylamine, pyrrole, indole and opiate derivative concentration determination method and a test kit for concentration determination, which provide fast, low-cost and low-effort quantitative detection of indole derivatives, in particular of psilocybin, and of arylalkylamine, pyrrole or opiate derivatives and in so doing enable precise determination of the presence and precise concentrations of different natural substances, such as psilocybin, for example, which have an indole as a structural fragment, and similarly structured substances having primary or secondary amines or pyrrole compounds in a rapid test. Said problem is solved in that the method comprises two method steps in the form of an extraction step and a subsequent analysis step using a color reagent, wherein in the analysis step at least one color reagent having at least one arylalkyl, pyrrole, indole or opiate derivative causes a quantitative and linear color reaction which is measurable by means of colorimetric methods and which is detected and evaluated.

The invention relates to an arylalkylamine, pyrrole, indole and opiate derivative concentration determination method and a test kit using this method for the exact determination of the presence and precise concentration of these substances (including e.g. psilocybin), which have an indole moiety as a structural element as well as similarly structured substances with primary or secondary amines or pyrrole compounds.

Psilocybin-containing mushrooms (so-called magic mushrooms) are a group of psychoactive mushrooms also known as magic mushrooms or hallucinogenic mushrooms. Mushrooms belonging to this group, such as Psilocybe cyanescens, also called “wavy cap”, contain the psychedelic (mind-expanding, hallucinogenic) substances psilocybin and psilocin, which are being researched as active ingredients.

WO 2019/073379 A1 discloses the large-scale production of psilocybin for use in medicine. In particular, it relates to a process for obtaining highly pure crystalline psilocybin, especially in the form of polymorph A. It further relates to a process for the preparation of psilocybin and intermediates in its preparation and psilocybin-containing formulations.

WO 2018/135943 A1 discloses the use of one or more cannabinoids and/or terpenes in combination with psilocybin and/or psilocin for use in the prevention or treatment of psychological or brain disorders. Preferably, the one or more cannabinoids selected from the group consisting of Cannabidiol (CBD); Cannabidioic acid (CBDA); Tetrahydrocannbidivarin (THCV); Tetrahydrocannbidivaric acid (THCVA); Cannabichromene (CBC);

Cannabichromic acid (CBCA); cannabigerol (CBG) and cannabigerolic acid (CBGA).

WO 2018/148605 A1 encompasses new compositions and methods comprising a psilocybin derivative. In one embodiment, the compositions disclosed herein before mentioned are used for a method of regulating a neurotransmitter receptor, e.g., a serotonin receptor. In one embodiment, the compositions disclosed comprise purified compounds, e.g., a purified psilocybin derivative, a purified cannabinoid, or a purified terpene.

WO 2019/079742 A1 encompasses methods and systems for increasing the safety of psychedelic drug therapies (e.g., 5-HT2A agonists, such as LSD and psilocybin), e.g., in the context of complex therapy. This includes, in particular methods and systems for reducing the risk of psychosis, hypomania, or mania associated with psychedelic therapy.

As a matter of fact, it has long been known that a solution of 2 dimethylaminobenzaldehyde in 20% hydrochloric acid, known as Ehrlich reagent, or Ehrlich-Pröscher reagent, is used to detect primary amino groups, pyrrole and indole derivatives. In pharmacy, the Ehrlich reagent is used to detect pyrrole, and indole derivatives, such as parent alkaloids. This reaction is also known as the Van-Urk reaction [S. Ebel and H. J. Roth (editors): Lexikon der Pharmazie, Georg Thieme Verlag, 1987, p. 213 and Ehrlich, P.: Über die Dimethylamidobenzaldehydreaktion (About the Dimethylamidobenzaldehydreaction).

The medical week and balneological central newspaper (1901) 151-153 and Urk, H. W., A new sensitive reaction for the ergot alkaloids, ergotamine, ergotoxine and ergotinine and its adaptation to the examination and colorimetric determination of ergot preparations. Pharm. Wochenblatt. 66 (1929) 473-481.]

In microbiology, dimethylaminobenzaldehyde is used as the Kovacs reagent for the detection of indole. This indole test is also suitable for the detection of lysergic acid diethylamide (LSD). For this purpose, the drug is mixed with a reagent solution consisting of Ehrlich's reagent, sulfuric acid and ferric chloride [Dibbem, H. W., Rochelmeyer, H., Studies on the Van Urk's color reaction of beta-. substituted indoles. Drug Research 13 (1963) 7-16.].

DE 29 35 881 C2 describes a device for detecting traces of marijuana. The detection reaction used is a color reaction to the marijuana ingredient (cannabis). The device consists of a cotton swab stored in a transparent plastic tube. The reagents required for the detection of cannabis are contained in separate reagent chambers of the plastic tube. The cotton swab is used to wipe the object or person to be tested, with traces of marijuana possibly accumulating in the cotton swab. This cotton swab is then dipped into the reagent solutions provided in the storage tube in a predetermined order. The detection reaction is based on a purely chemical reaction of the cannabis molecules with the reagents provided in the device at a defined pH value. In the presence of cannabis, a color reaction is seen in the final solution.

EP O 229 517 A1 describes an absorbent, reagent-impregnated test paper for the detection of illicit drugs or their metabolites in biological fluid samples. According to the technical solution of EP O 229 517 A1, the sample liquid has to be pre-treated to detect the drugs. For this purpose, it is chromatographically purified and concentrated by means of specially prepared syringes.

After the pretreatment, the sample is loaded onto the sample application zone of the test paper. From there, the sample liquid migrates to a reagent-impregnated zone where the detection reaction for the target analyte takes place and can be observed by means of a color reaction.

This is also a purely chemical detection reaction. It is known that the detection sensitivity and specificity of such reactions are not sufficient to detect drugs in body fluids.

Therefore, according to the technical solution of EP O 229 517 A1, a step for concentration and purification of the sample is carried out upstream.

The complex nature of the sample preparation and reaction control in the detection reaction on the test paper makes the detection method described in EP O 229 517 A1 appear unsuitable for use “on site” and in particular, for use by “laymen”.

DE 101 11 224 A1 encompasses a system for improved detection of compounds of the ecstasy class in biological samples, wherein new analogs of the ecstasy class are provided for the detection of such drugs. These analogs are compounds, or salts thereof, of a 2-aminomethylenedioxyphenyl (MDP) derivative attached to Z, wherein Z is a moiety capable of binding either directly or indirectly to an immunogenic carrier, a detectable label, or a solid capture vehicle. Such analogs can be used to construct immunogens, enzyme or enzyme-donor conjugates, and other conjugates.

The immunogens produce reproducible antibodies with an excellent ability to distinguish various ecstasy classes drugs from potentially interfering substances in biological samples. The specific antibodies and the conjugates can be used to distinguish and measure various ecstasy class compounds in biological samples such as those obtained from an individual suspected of substance abuse.

DE 43 05 593 C1 encompasses a test system for the colorimetric detection of drugs, in particular of opium derivatives, cocaine and amphetamines. In this context, an easy-to-use test kit is provided which minimizes the disadvantages of known solutions with regard to possible hazards to the personnel in charge of the test due to cuts, burns and deflagrations as well as with regard to possible environmental pollution. According to DE 43 05 593 C1, the test system consists of an ampoule, a carrier material and a solution of a color reagent, the carrier material being non-porous and the weight ratio of carrier material to color reagent being within the limits of 100 to 5 to 20. This achieves a significant reduction in the proportions of reagent and solvent required.

Since the discovery of the psilocybin molecule in 1959 to the present time, the concentration determination of psilocybin and similar other substances has not evolved significantly and represents a costly and lengthy procedure. There is a need for a substantial simplification of the test methods, which would allow their widespread use. Many data on occurring ingredients in mushrooms, plants or animals have already been inaccurate for many decades, even in the technical literature, and are based on individual measurements, also local and seasonal variations are not taken into account. As an example, to mention: the mushroom Pluteus salicinus whose Psilocybin content is indicated in the technical literature with 0.1 to 0.8%, there are however contradictory analyses of Gartz who suggests a content of over 2%. This confusion and also misinformation prevails with many natural substances which are consumed from mushrooms or plants, and are based on the limited possibility to determine the concentration of these.

A simplified test system for this concentration determination is desirable to further improve the previous findings, and it would be very good if this test system could also serve to screen for other mushrooms containing the active ingredient psilocybin that were not previously tested because they were considered toxic or inedible. The active ingredient psilocybin has already been discovered in over a hundred species of mushrooms around the world, and new species are being added all the time.

Currently, the determination of the concentration of indole derivatives, such as psilocybin, which is produced by fungi of the genus Psilocybe, among others, still requires complicated and extensive analysis in special laboratories. The fruiting bodies of the mushrooms have to be sent to a special laboratory where the active ingredient psilocybin is extracted by means of multi-stage and complex protocols with the aid of a wide variety of solvents.

In order to illustrate the time and cost required to look for such active ingredient quantity determinations, the steps currently used for such a detection reaction of psilocybin from the fruiting bodies of the fungi of the genus Psilocybe are shown below.

The extraction starts with the drying of the fruiting bodies by means of freeze-drying, whereby larger fruiting bodies may require several days to dry. In the next step, the fruiting bodies are ground to a fine powder using a mortar, and methanol is added to extract the active ingredient psilocybin while stirring continuously for several hours.

The biomass of the fungus is subsequently separated by vacuum filtration. To ensure that all psilocybin is extracted with the methanol, this step must be repeated at least three times.

The resulting methanol extract is combined in a large glass flask and concentrated using a rotary evaporator.

The following steps involve purification using different solvents, such as cyclohexane, in a separating funnel, which is used to remove the fats from the extract.

Centrifugation and filtration are used to remove the last small particles that could clog downstream machinery.

After a clean extract has been obtained, the amount of psilocybin present can be compared to a previous accurate measurement of a laboratory standard using high performance liquid chromatography (HPLC).

The difficulty here is that psilocybin, as a very polar molecule, is difficult to separate with the commonly used standard C18 column material. Some optimization of the standard methods is required to achieve a meaningful separation of the different indole derivatives.

This is necessary because baseline separated individual peaks are always required for the calculation of the amount of active ingredient. Currently, even the availability of pure psilocybin is severely limited and only very few laboratories worldwide even have an internal standard for performing such measurements. In the evaluation, therefore, a wide variety of calculations including the measured laboratory standard must be carried out in order to be able to conclude on the quantity of the present constituents.

The second way to obtain the content of derivatives from biological samples is the targeted extraction and purification of these substances.

For this purpose, various extraction and purification steps are combined and the extracted substances are prepared for analysis by means of multiple purification over different columns using HPLC and recrystallization for final purification and confirmation of substance purity via nuclear magnetic resonance spectroscopy. Afterwards, the obtained purified psilocybin can be weighed from the defined amount from which extraction was performed.

However, this method gives very inaccurate results because a large portion of the psilocybin is lost during purification. This is due to the property of the psilocybin molecules, which are destroyed when high pressure and temperatures are used, or which remain in the individual purification steps.

The following remarks on the possible determination of the concentration of psilocybin are also intended to explain in more detail the currently possible methods for the determination of many similar substances, which all require similarly complicated purification and analysis methods.

The determination of the concentration of other indole or pyrrole derivatives or substances with primary amines and similar substances from biological samples also always require a multi-step extraction, which is adapted according to the type of sample.

A very clean extract must always be produced in order to avoid contamination of the highly sensitive measuring instruments used in the subsequent analysis.

As with the example of psilocybin, the analysis can be carried out not by HPLC, but also using gas chromatography or similar methods. However, all these methods have in common that samples are first sent to a competent special laboratory with the respective authorizations, where they have to be extracted, analyzed and evaluated in a time-consuming manner before a statement can be made about the concentration of the natural substances contained.

The entire preparation and analysis process is very time-consuming and, above all, very cost-intensive, which is why it has so far only been used, for example, by authorities for the analysis of drug finds. There is practically no possibility of determining the concentration of psilocybin, for example, in mushrooms found in the wild outside these structures.

Another disadvantage is that there is currently no mobile rapid test for the concentration determination of pyrrole, phenylethylamine and indole derivatives.

Previous tests could only indicate the presence of the substances, but they do not provide any information on the concentrations of those samples.

In biological materials, for example, the concentration of pyrrole, phenylethylamine and indole derivatives varies greatly. For example, psilocybin can be formed in fungi in varying amounts seasonally, and each of the more than one hundred species reaches concentrations ranging from 0.1% to 3%. Thus, if the specific species of one and the same genus are confused, there is a high risk of accidental overdose.

Another major disadvantage of these methods used to date is the lack of rapid tests for urgent medical use. These would be very desirable, for example, for patients who are admitted for treatment with a strong presence of confusion and are suspected of mushroom poisoning or overdose.

The rapid analysis of body fluids by means of a rapid test by the attending physician could in this case very quickly provide a cause for the observed symptoms, which would enable the prerequisite for a necessary targeted and effective treatment.

Especially in patients with overdoses of natural substances of this kind, correct treatment is immensely important for the subsequent mental health state, since incorrect care by misjudging a drug-induced psychotic episode can immensely aggravate the symptoms of this state and enable permanent psychotic illness.

Monitoring of this kind by means of a rapid test would also quickly confirm the suspicion of consumption of mind-altering substances in the event of traffic or other checks by the state, and, thanks to the determination of concentrations, also permits conclusions to be drawn about current influences at high blood concentrations or about the dosage and time of consumption using urine samples.

The problems listed also occur in the area of other natural substances. For example, when LSD is purchased, which is usually dripped onto a small piece of paper, reliance must be placed on the dealer's indication of the amount of active ingredient, which can just as quickly lead to undesirable overdoses and side effects. In many cases, it is not even the molecule that is actually desired and advertised accordingly.

The same problem arises in the production of N,N-dimethyltryptamine (DMT)-containing extracts from plants of the genus Psychotria, whose indole derivative is obtained by boiling the leaves of the plant for several hours with unknown active ingredient content.

DMT can be consumed as a pure substance or mixed with MAO inhibitors such as the Harman alkaloids (also a measurable derivative) to make a potent ritual brew called ayahuasca. This drink has great cultural significance in many indigenous cultures of the Amazon region, and the ceremonial use of the mixture has become increasingly popular globally, especially in the last decade. So-called Ayahuasca Retreats through companies such as “Ayahuasca International” offer ceremonies worldwide, but these do not meet the standards of care and concern for the individuals undergoing the ceremony, nor can they provide an estimate of the active ingredient content of the mixture. Therefore, the risk reduction through an exact determination of the amount of the active ingredient DMT through a rapid test kit would be essential in order to contain the risk of traumatizing experiences through inaccurate dosing as far as possible from the beginning.

Recent studies further show extremely promising results in applications of psilocybin for patients with terminal cancer existential crises, treatment-resistant depression, or addictive behaviors (Johnson and Griffiths 2017). Phase 2 studies conducted by Johns Hopkins University and New York University showed treatment success of 60-80% for previously treatment-resistant depression and anxiety in patients (Sherwood and Prisinzano 2018). These results are only exemplary in that clinical trials already exist for other derivatives already discussed, and these compounds are being tested for use within a wide variety of therapeutic domains.

In summary, there is still no way to easily determine the concentrations of arylalkylamine, pyrrole, indole and opiate derivatives to avoid extremely time-consuming and costly methods, such as exact laboratory analysis.

The task of the invention is to avoid the disadvantages of the prior art and to provide an arylalkylamine, pyrrole, indole and opiate derivative concentration determination method as well as a test kit for the concentration determination of these substances, which allows a fast, cost- and effort-efficient quantitative analysis, and effort-saving quantitative detection of indole derivatives, in particular of psilocybin, as well as of pyrrole or arylalkylamine derivatives or opiate derivatives, and thereby provide an exact determination of the presence and precise concentrations of various natural substances, such as, for example, psilocybin, which can be used to determine the concentration of a natural substance. Psilocybin, is which has an indole as a structural fragment, and similarly structured substances with primary or secondary amines or pyrrole compounds or opiates in a quantitative rapid test.

This task is solved by a method according to the first patent claim and by a kit according to the seventh patent claim. Advantageous embodiments of the invention are given in the subordinate claims.

The essence of the invention is that the color reaction, which is now known for more than a hundred years, is used specifically for a measurable quantitative and linear color reaction of arylalklyamine, pyrrole, indole and opiate derivatives in a two-step method for determining their concentration.

This two-step concentration determination method starts with a defined extraction of the respective derivatives from the sample, which can be very diverse and is known from the prior art.

Subsequently, in the second stage of the process, the extract obtained in this way is subjected to a quantitative determination of the concentration of the respective active ingredient by means of colorimetric methods, thus enabling precise information to be obtained on the active ingredient content present.

This simple procedure enables a rapid determination of the content of relevant pyrrole, phenylethylamine and indole derivatives from biological materials, such as fungi and plants, or from synthetic products, which is the basis for a test kit for rapid quantitative tests.

The provided test kit allows a concentration determination of psilocybin, psilocin, LSD, DMT, and further indole derivatives, primary aromatic amines, especially tryptamines, such as e.g. NMT, 5-MeO NMT, dimethyltryptamine, 5-MeO-DMT, 5-bromo-DMT, bufotenine, N-methylserotonin, serotonin, psilocin, psilocybin, baeocystine, norbaeocystine, melatonin, tryptophan, AMT, 5-MeO-AMT, AET, NET, DET, DiPT, DPT, DBT, 4-HO-MET, 4-HO-DET, 4-PO-DET, 4-HO-DIPT, 4-HO-MiPT, 5-MeO-DALT, 5-MeO-DiPT, or sumatriptan, and phenylethylamines, such as. Amphetamine, methamphetamine, ephedrine, pseudoephedrine, norephedrine, norpseudoephedrine, oxilofrine, N-methylephedrine, N-ethylamphetamine, PMA, PMMA, methyl-MA, 2C-B, 2C-C, 2C-D, 2C-E, 2C-P, 2C-I, 2C-F, 2C-N, 2C-T-2 2C-T-4, 2C-T-7, 2C-T-8, 2C-T-9, 2C-T-21, 2C-TFM, MDA, MDMA, MDEA, MDE, MMDA-3a, MMDA, Mescaline (M), IM or TMPEA, pyrroles and similar substances using a single assay procedure that is rapid, low cost and low effort.

Due to the simplicity of the procedure and the simple construction of the test kit, as well as the relative harmlessness of the chemicals used, even inexperienced laymen can determine the active ingredient content of, for example, mushrooms found in the wild or substances purchased from an unknown source with the help of precise instructions.

In this way, the medical applicability of these substances as well as the safe consumption of these pharmaceutically quite interesting substances can be ensured, important findings in the acute treatment of poisonings and overdoses can be obtained or, however, criminally relevant active substance quantities can be determined on the part of the state.

Due to the simple test procedure, the test kit can be produced in large numbers for a low price, which allows the large-scale use in the scene of “Safer Drug Use” within already existing projects for risk minimization in drug use, for later use in psychotherapy against depression by psychologists or alternative practitioners or finally also for private use by mushroom pickers or growers.

The invention is explained in more detail below with reference to the exemplary embodiments and the figure, without being restricted to these. It shows:

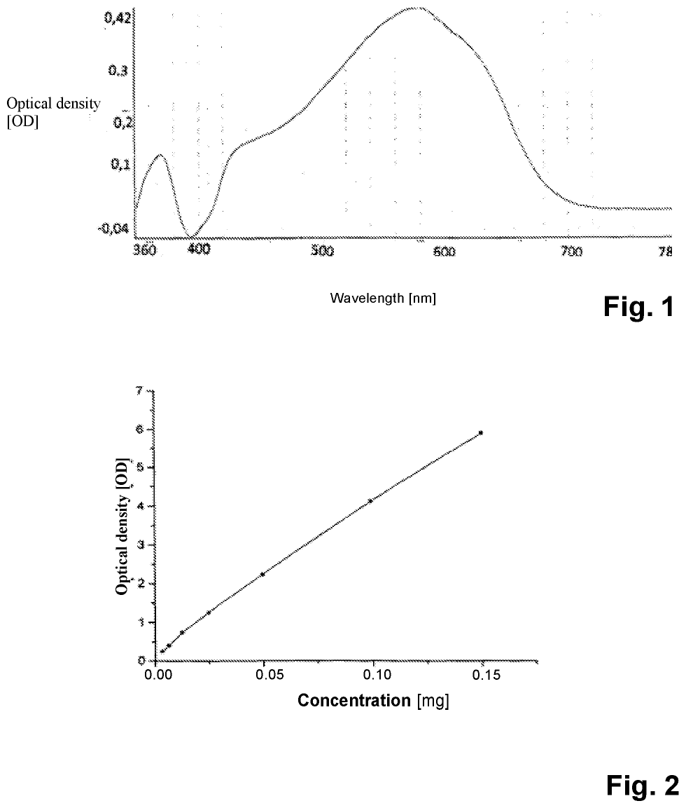

FIG. 1: A diagram of the absorption measurement (optical density/wavelength) to determine the absorption maximum of psilocybin,

FIG. 2: A diagram of the optical densities of a dilution series of a psilocybin standard after staining by means of an embodiment of the method according to the invention for determining the concentration of psilocybin,

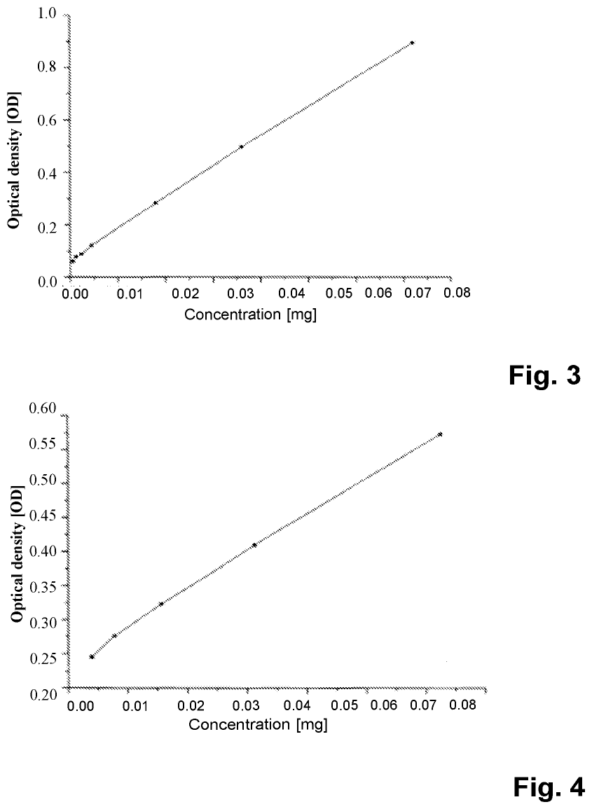

FIG. 3: A diagram of the optical densities of the dilution series of a DMT standard after staining has been carried out by means of an embodiment of the method according to the invention for determining the concentration of DMT,

FIG. 4: A diagram of the optical densities of the dilution series of an LSD standard after staining by means of an embodiment of the method according to the invention for determining the concentration of LSD

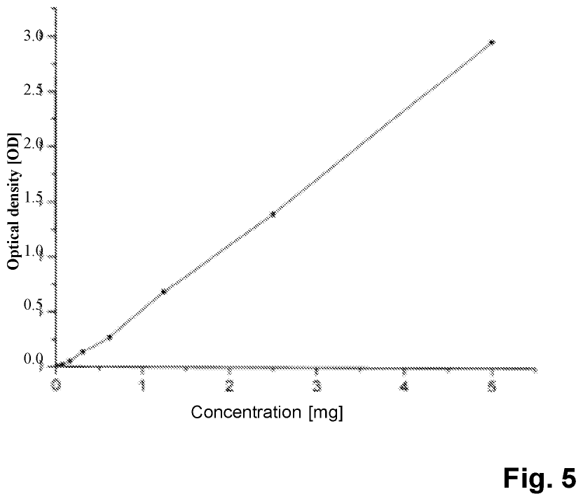

FIG. 5: A diagram of the optical densities of the dilution series of an amphetamine standard after staining by means of an embodiment of the method according to the invention for determining the concentration of amphetamine.

The test procedure, which forms the basis for the test kit, consists of two procedural steps to determine the concentration of arylalkylamine, pyrrole, indole and opiate derivatives, particularly in the form of psilocybin, DMT, Harman alkaloids and their related derivatives, carbazoles as well as other substances with primary amines, parent alkaloids or ergotamine as well as synthetically produced lysergic acid derivatives such as lysergic acid diethylamide (LSD), drugs or substances with a similar structure.

The test method can also be used to detect substances in which primary aromatic amines or similar substances are only formed by a reaction such as after hydrolysis. These variously listed substances can also be found in dried fruit bodies, mycelia, sclerotia or other plant or animal materials, in body fluids such as urine or blood, in foodstuffs, wastewater or synthetic products of any origin.

Furthermore, the test method is also used to measure the concentration of bacterial cultures or other organisms containing the enzyme tryptophanase, for example, and allows conclusions to be drawn about the concentration and growth of these bacteria. For each derivative, the two-step test procedure for concentration determination is individually adapted, with the respective test kit containing the necessary materials and instructions for extraction and subsequent concentration determination.

In the first step of the process, the active ingredient is extracted or absorbed in a defined quantity of water and/or organic solvent, which, depending on the substance, may be an aqueous acidic solution or an alcoholic solvent or other similar solvents. This is done according to the prior art.

In the second, subsequent process step, a defined amount of solvent containing the active substance is removed and the dye solution is added. The appropriate solvent is selected according to the known prior art.

The staining solution for this method comprises, for example, the electrophilic 4-(N,N-dimethylamino)benzaldehyde (abbreviated: DMABA).

For the concentration determination of psilocybin, the staining solution contains, for example, the electrophilic 4-(N,N-dimethylamino)benzaldehyde (DMABA) in a hydrochloric acid aqueous solution for the staining of psilocybin.

Depending on the active ingredient, the DMABA can also be dissolved in other solvents or be in powder form, or contain other additives such as other acids or solvents or metal ions such as Fe³⁺. The selection is made according to the known state of the art.

In a reaction of the DMABA with indole compounds and the substances listed above, strongly colored compounds are formed, which are optically detected and evaluated. The measured values are compared with a curve of measured values obtained by calibration, so that a quantitative concentration determination is made by this comparison on the basis of the color intensity of the test substance present.

Due to the high reactivity of DMABA, it is also possible to stain substances that do not have a nitrogen atom/amine function.

If the second process step is carried out at an elevated temperature compared to the ambient temperature, which is useful for psilocybin, for example, the required reaction time is significantly reduced. The required heat supply varies depending on the substance and can be achieved in an oven.

The staining solution for this procedure includes 4-(N,N-dimethylamino)-benzaldehyde or alternatively the following reagents:
Marquis reagent, (which is classically used for the detection of various alkaloids, such as morphines or various phenylethylamine derivatives, and is based on the use of sulfuric acid and formaldehyde),
Mecke reagent (which also dyes alkaloids such as LSD, morphine and many other drugs),
Zwikker reagent (which is used for quantitative detection of mainly barbiturates and other drugs),
Simon/s reagent (which is also mainly used for the detection of alkaloids with secondary amines and others and also has a linear color gradient and is used in combination with Mecke and Marquis for the identification of alkaloids)
Mandelin reagent (which is mainly used for the detection of ketamine and other drugs),
Liebermann Reagent (which also detects classical alkaloids and other active ingredients. Usually used for the detection of cocaine and morphine),
Fröhde's reagent (which is usually used for the detection of opiates as well as other active substances),
Folin's Reagent (which is used for the differentiation of MDMA and related compounds),
Dille-Koppanyi Reagent (which is mainly used for the detection of barbiturates),
Scott Reagent (which is used for the detection of cocaine and other drugs) and
Duquenois-Levine Reagent (which is used for the detection of cannabinoids).

The reagents listed above showed different staining with the substances to be quantitatively tested, which gives a good possibility to distinguish the different substances to be tested, allowing also mixtures of substances to be quantitatively tested to determine the different concentrations, since all of the listed reagents show a linear course of the concentration curves in the absorption measurements of the color reactions (shown by way of example in FIGS. 2 to 5), so that these reagents, which have been known for a long time, can also be used for a determination of the active substance concentration by means of the method for indole alkaloids, phenylethylamines and other compounds and can be used for the colorimetric determination of active substance concentrations for use in the test kit.

The observed color change of psilocybin/psilocin can be described as e.g. violet to bluish in higher concentrations, whereas the discoloration of DMT can be observed e.g. from yellow to green in higher concentrations. The color of LSD together with the dye solution can be described as brownish. Depending on the solvents used, such as the addition of methanol, the observed color may vary. This is shown in FIGS. 6 and 7 (=the photos).

The specific optimal incubation temperatures of the individual reagents are included in the enclosed user instructions of the respective test kits.

For mobile use, the use of latent heat storage or the simple use of batteries or accumulators for the generation of the required heat as well as other possible heat sources is advisable.

It is also possible to couple the detection reaction to an exothermic reaction, which provides the required heat supply, or instead to use metal ions, such as Fe³⁺ ions to support the reaction.

Surprisingly, the coloration in the second reaction step shows a linear range within which an exact determination of the concentration of the active ingredient (pyrrole, phenylethylamines and indole derivatives, opiates, cannabinoids) is possible.

By using a calibration specific to each active ingredient, the process compares and determines the exact active ingredient content on the basis of a comparison of the color intensities with a determined color spectrum of an exact dilution series and the associated active ingredient contents.

This optical comparison during the second step of the process allows a fast and direct determination of the concentration of the tested sample. The necessary evaluation of the test result can be compared with the color reaction of a pH test strip and the reading of the pH value on a color scale.

This evaluation of the test procedure for measuring the concentration of various substances can, however, also be measured by means of technical auxiliary devices or other procedures, such as a spectrophotometric analysis, and thus allows an even more exact determination of concentration at values which lie exactly between 2 colors in the color scale.

An improvement of the purely optical evaluation, which is possible with the human eye by using a color scale, is achieved with the aid of a modern camera phone (smartphone) by means of an exact color determination by the camera of the cell phone.

By means of an APP (application software) and reference values, a more exact determination of the concentration than with the human eye can be made very easily in this way.

Within the scope of the invention, there is also a combined test system in a closed vessel, bag or other cavities, which already contains the required extraction solution and the required color reagent, such as DMABA) and possibly other substances or solutions in a separate ampoule or other types of cavities. By adding the test substance and releasing the staining solution, the concentration can also be determined after the incubation has taken place. These “single or multiple kits” are particularly interesting for fast, uncomplicated and, mobile concentration determination by authorities, in hospitals and many other areas of application.

The complete test kit comprises “single-use kits” as a single-use system or the “multiple-use kits” as a multiple-use system as well as user instructions. The handling of the test kit and the specification of the active substance-specific optimum incubation temperatures are included in the enclosed user instructions.

This test kit for the determination of concentrations enables a fast, cost- and effort-efficient quantitative detection of indole derivatives, in particular of psilocybin, as well as of pyrrole or phenylethylamine derivatives, whereby an exact determination of the presence and precise concentrations of various natural substances, such as psilocybin, which possess an indole as a structural fragment, as well as similarly structured substances with primary or secondary amines, pyrrole compounds, cannabinoids, opiates or barbiturates is possible in the rapid test.

### Design Example 1

Dried fruiting bodies of fungi of the genus Psilocybe are crushed to a fine powder by means of a mortar, blender or other means and a defined quantity of 1 g is provided by means of a balance or other suitable measuring equipment.

The biomass is placed in a suitable container such as a 100 ml beaker and soaked with the 5% citric acid provided and incubated for a short time at room temperature.

In the following step, the sample beaker is filled with 50 ml of distilled water and the extraction is carried out with vigorous shaking. Using the syringe also included in the test kit, exactly one milliliter of the extraction solution can now be taken, preferably without fungal biomass.

For an even more precise measurement a filtration is recommended, which can be achieved, for example, by using an attached syringe filter, but is not absolutely necessary.

The next step involves incubation with 3 ml of detection solution containing a defined amount of 40 mg/ml p-dimethylaminobenzaldehyde (4-(CH₃)₂NC₆H₄CHO) in an aqueous hydrochloric acid solution. This results in a reaction with the p-dimethylaminobenzaldehyde and an intense blue-violet coloration depending on the additives used in the staining solution and the concentration of ingredients present.

If the concentration is too high, an opaque violet-blackish color will result, which determines the detection limit of the reaction, since no optical differences can be detected. In this case, the respective instructions must be followed exactly, which means that the amount of extraction solution must already be adjusted to the expected amount of ingredients. If the concentration of the substances to be detected is too high, it is recommended to dilute or reduce the selected extraction solution beforehand, with an adjustment to the use of the amount of detection solution.

This is done with regard to the subsequent measurement, which has its linear range only in a certain area and in which a color change is optically visible.

The color change occurs, for example, in the reaction of psilocybin with DMABA to form a triarylmethane dye according to the following reaction scheme:

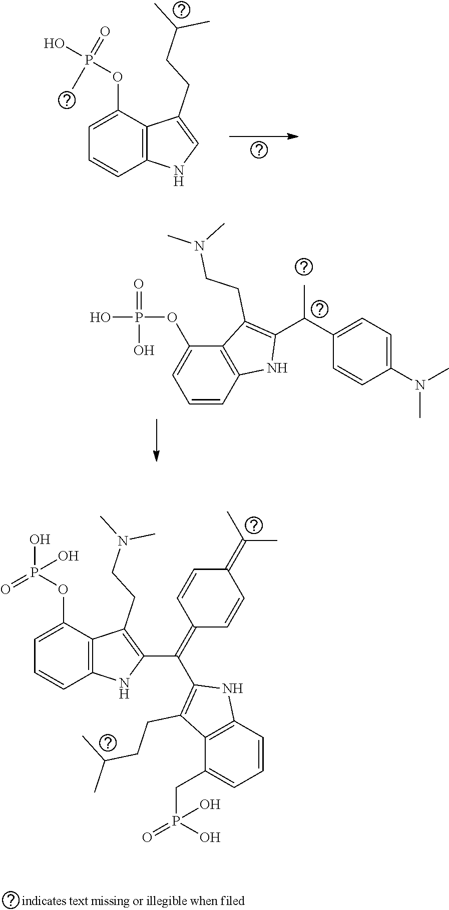 [SMILES: CC(C)CCc1c[nH]c2cccc(OP(C)(=O)O)c12 ; CC(c1ccc(N(C)C)cc1)c1[nH]c2cccc(OP(=O)(O)O)c2c1CCN(C)C ; CC(C)=c1ccc(=C(c2[nH]c3cccc(CP(=O)(O)O)c3c2CCC(C)C)c2[nH]c3cccc(OP(=O)(O)O)c3c2CCN(C)C)cc1]

The absorbance measurement is shown in FIG. 1 and shows a maximum absorbance of 550 nm, which is consistent with the observed color.

### Design Example 2

A dilution series is prepared from an authentic psilocybin standard, which was also incubated with the dye solution described above and measured at a wavelength of 550 nm. The result of the very linear color reaction is shown in FIG. 2 and provides the basis for a quantitative concentration determination.

Different dilution levels and their observed color curves serve as a basis for a colorimetric determination of unknown psilocybin concentrations, as described in the example above. By means of precise calibration, the exact concentration of psilocybin and thus the active ingredient content in the mushrooms can be determined by comparing the color gradients and shades of color.

### Design Example 3

DMT is usually purchased by consumers as a light-colored crystalline or powder substance. This is usually the product of a simple extraction of DMT-containing plants such as Psychotria viridis. To determine the actual drug content of DMT in this powder, 10 mg of the substance is weighed and dissolved using 1 ml of acidified water (4% HCl solution in Aqua bidest). By adding 3 ml of staining solution with p-dimethylaminobenzaldehyde in a hydrochloric acid aqueous solution, the color reaction is carried out with incubation at 50° C. for 15 minutes. Subsequent measurements determined the maximum absorbance of the resulting color reaction at 472 nm. The absorbances obtained can now be compared or the resulting color can be compared colorimetrically with the colorations of an authentic standard series.

This comes from a standard from which a dilution series was prepared and examined with regard to a linear color gradient. The results can be seen in FIG. 3 and also show a linear color gradient for DMT, which forms the basis for a colorimetric determination of the concentration.

The measured absorbance or the observed color gradient of the sample can also be assigned by means of the compensation lines and the already known concentration.

### Design Example 4

Due to the small amount of LSD used for consumption, the substance is often found on absorbent cardboard (blotter). This small piece of cardboard is placed in a 1.5 ml Eppendorf reaction vessel and 0.5 ml of pure ethanol is added.

With shaking, the extraction of the LSD is completed. The ethanol can be removed and incubated with 1.5 ml of staining solution containing p-dimethylaminobenzaldehyde in a hydrochloric acid aqueous solution at 50° C. for 15 minutes.

The resulting color can now be compared colorimetrically with the color of previously known amounts of drug. A dilution series of an authentic standard and the subsequent staining of the individual samples also again show a linear color progression with increasing concentrations (see FIG. 4). The samples are measured at the maximum absorbance at 530 nm. The unbelievably high sensitivity of the test is remarkable even at a few micrograms of LSD and thus also proves the function of the concentration determination in the group of ergot alkaloids.

### Design Example 5

Amphetamine is usually purchased by consumers as a light crystalline or powder substance. To test how much active substance is present in this powder, 10 mg of the substance is taken and dissolved using 1 ml ethanol. By adding 3 ml of dye solution containing p-dimethylaminobenzaldehyde in a hydrochloric acid aqueous solution, the color reaction occurs with incubation at 50° C. for 15 minutes. By a subsequent measurement, the maximum absorbance at 510 nm was determined. The obtained absorbance value at 510 nm or the resulting color can now be compared colorimetrically with the colorations of an authentic standard series. A dilution series was prepared in advance from the standard and examined with regard to its linear color progression.

The results can be seen in FIG. 5 and also show a linear color gradient for amphetamine, which forms the basis for a colorimetric determination of the concentration. The measured absorbance or the observed color gradient of the sample can also be assigned by means of the compensation lines and the already known concentration. It is evident that likewise the concentration of phenylethylamine derivatives such as amphetamine and many other examples not shown can be determined with high sensitivity and simple methods by means of colorimetric methodology.

The method and the test kit allow the measurement of the concentration of pyrrole and indole derivatives, such as psilocybin, N,N-dimethyltryptamine (DMT), other active ingredients, such as e.g. 5-methoxy-N,N-dimethyltryptamine (5-MeO-DMT) and similar indole alkaloids, 5-MeO-DIPT, 5-MeO-MIPT, 5-MeO-AMT, 4-AcO-DMT, 2C-D, 2C-E, 2C-I, 2C-P and DXM, the Harman alkaloids and other derivatives, carbazoles, cannabinoids, opiates and other substances with primary or secondary amines, ergot alkaloids and ergotamine as well as synthetically produced lysergic acid derivatives, such as e.g. Lysergic acid diethylamide (LSD), drugs, other semi or fully synthetic products or substances with similar structure. These substances have a wide distribution and can occur in dried fruit bodies, mycelia, sclerotia or other plant or animal materials, in body fluids such as urine or other, in food or other products of synthetic origin. Furthermore, the detection reaction can also be used to measure the concentration of bacterial cultures or similar, which contain, for example, tryptophanase.

Dilution series with Marquis reagent have also shown a linear color gradient. The Marquis reagent stains DMT linear brownish/yellow. For this purpose 5 ml of concentrated sulfuric acid was mixed with 250 μl 37% formaldehyde. DMT and psilocybin are present in an aqueous 3.5% HCL solution and the same amount of Marquis reagent was added. In the last row the 3-fold amount of reagent was used which showed a stronger coloration. In general, it can be summarized that many substances whose concentration can be detected by DMABA can also be determined by Marquis reagent, in lower concentrations of the detection solution, the substances can also be well characterized from each other by their color differences. The Marquis test also shows linear color gradients measured at 450 nm and can therefore be used for colorimetric determination. Based on this, other color tests such as Mecke, Simon, Liebermann, Folin, Zwikker Mandelin, Froehde and many more have also been investigated and confirmed for their linearity. These additional color tests are also suitable for the concentration determination of the mentioned indole alkaloids, pyrrole and phenylethylamines as well as by the extension of the color test to other detection methods now also for the detection of opiates and cannabinoids.

The method and the test kit are used for the detection of substances in which primary aromatic amines or similar substances are formed by upstream hydrolysis, which can be detected with this test method. The concentration is determined by a simple color reaction, which can be evaluated purely optically by means of colorimetric evaluation or with the aid of measuring instruments.

The application of a mobile test kit for the above-mentioned derivatives can, if necessary, be carried out under heat supply, which by means of an external heat source enables a concentration determination independent of outside temperatures or additional power source.

It is also within the scope of the invention that the test kit is in the form of test strips with an applied color reagent, advantageously 4-(N,N-dimethylamino)benzaldehyde, Marquis reagent, Mecke reagent, Zwikker reagent, Simon's reagent, Mandelin reagent, Liebermann reagent, Fröhde reagent, Folins reagent, Dille-Koppanyi reagent, Scott reagent or Duquenois-Levine reagent, (whereby the test strip can be obtained according to the state of the art for the production of test strips), which can be dipped directly into liquids, whereupon by means of the optically evaluable color reaction a simple and uncomplicated concentration determination takes place according to the method, in particular the second step of the method, the user has a very easy-to-handle system, which is processed similarly to the test strip system for pH value determination. The provision of a rapid test kit for single-use, in which the required solvent is already contained in a bag or similar storage object, and into which the respective sample must subsequently be added, enables simple handling by the user, who may even be a layman.

The detection solution is added manually or is already contained in a breakable ampoule or similar container, in which the color reagent, preferably 4-(N,N-dimethylamino)benzaldehyde, Marquis reagent, Mecke reagent, Zwikker reagent, Simon's reagent, Mandelin reagent, Liebermann reagent, Froehde reagent, Folins reagent, Dille-Koppanyi reagent, Scott reagent or Duquenois-Levine reagent and releases the color reaction by simply adding or breaking or similar the storage vessel.

After incubation, the presence and concentration of the above-mentioned substances is determined by optical evaluation using colorimetric methods, which are known per se.

The method and the test kit for the exact determination of the concentration of the above-mentioned substances can be applied in a micro-dosing area, e.g. in the form of a psilocybin micro-dosing kit, by means of which the required dose of 2-3 mg can be calculated exactly to the amount of mushroom used, whereby the prepared extraction solution consisting of water and citric acid can be used safely in order to achieve a specific dosage.

In the required case, it is also within the scope of the invention to use an external heat source for incubation during the procedure and when the test kit is used as intended.

The method and the kit enable:
the exact dosage of indole alkaloids used in psycholytic therapy and other substances for precise application in drug therapy and medicine,
the determination of the presence and amount of psychoactive indole alkaloids or other substances in wild-collected mushrooms and plants or any other materials, thus preventing dangerous mix-ups,
the precise determination of quantities of active substances in body fluids and tissues and other materials for the assessment of the need for medical intervention in the event of overdose symptoms or of the criminal liability of people and other applications,
the measurement of drug levels in organism cultures, such as cultures of bacteria containing tryptophanase,
the testing of (illegally acquired) substances for ingredients and their concentrations in combination of different tests to avoid poisoning, overdoses or undesired effects, e.g. to test for PMP/PMMA concentrations in ecstasy pills or powders,
as well as mobile measurement during field trials or similar.

The provision of the single-use test kit, which already contains the required solutions in different wells for the procedure and where only the substance to be tested needs to be added, also enables very simple, low-effort handling, even by non-experts such as patients and end users.

Possible areas of application for the method and the test kit include doping tests, impaired driving tests, poisonous mushroom selections, and substance and environmental monitoring.

The method and test kit can also be used for quantitative analysis of unknown street drugs in tablet form, such as ecstasy, which are often filled or stretched/blended, mixed with other substances.

Due to various stretching or filling substances, neither illegal drugs available on the black market nor legal industrially produced drugs in tablet form can be detected using standard colorimetric methods, since, for example, sugar or starch (common fillers) react with the detection reagents or components of these to form strongly colored compounds and thus mask the coloration of the actual active ingredient.

The mentioned fillers sugar and starch react for example with the sulfuric acid of the detection reagent to a brown color and the actual purple coloration of MDMA is thereby covered by the brown color.

This can be easily demonstrated experimentally, as shown below:

0.05 mg MDMA in 100 μl aqueous solution turns to a strong purple color by the addition of 200 μl of a mixture of 20:1 concentrated 97% sulfuric acid and 40% formaldehyde.

If only 0.10 mg of starch is added, the mixture turns brown when treated with the detection reagent mentioned above, thus obscuring the intended color reaction and making it impossible to evaluate.

To circumvent this problem, the quantitative detection method is used for substances such as MDMA, from the group of phenylethylamines (tropane alkaloids or opiates are rarely present as pure substances in illegal narcotics), which are often filled or stretched.

In addition to the quantitative detection of the desired active ingredient, unwanted stretching or filling substances are removed, which make it impossible to evaluate the color reaction.

In addition to the fillers, there is often a mixture with other active ingredients, which can sometimes have lethal effects on the consumer if the dosage is incorrect.

Here, too, the detection method provides a remedy by using different detection reagents matched to the expected excipients, as shown by the following example.

A possible case of lethal admixture is, for example, the substance PMMA (para-methoxy-N-methylamphetamine) in MDMA (3,4 methylenedioxy-N-methylamphetamine), also known as ecstasy.

The method allows the removal of unwanted fillers and the concentration determination of different active ingredients for a wide range of drugs with a set of color reagents without the use of, for example, expensive and only very specific antibodies. For the first time, it is possible to quantitatively analyze the active ingredient content of mixtures without the use of thin-layer chromatography.

Depending on the expected active ingredient, different color reagents are combined for this application, which also cover all previously used extenders. For example, for phenylethylamines in the following example, the detection of common extenders such as amphetamine or 2C-B is shown by means of a second staining specifically for these substances. For the detection of opiates and tropane alkaloids and their contained extenders, a combination of several color reagents is also used and enables the detection of e.g. fentanyl in heroin. However, the necessary removal of fillers before the actual staining of the expected active ingredient remains the same for these active ingredients, and this is made possible by removing them before the actual staining by mixing the color reagent 1:1 with chloroform. The actual dyeing is not affected by this treatment.

### Design Example 6

Quantitative detection of possibly stretched phenylethylanine, such as MDMA (also known as ecstasy) in tablet form.

For the test, 10 mg of powder of an unknown tablet, which is stretched/blended with fillers, is scraped off with a knife and weighed exactly with a precision balance.

Two detection solutions are prepared for the determination of the active substances present.

For the first detection solution, 400 μl chloroform is added to a 1.5 ml glass vessel with screw cap. To this is added 10 μl formaldehyde and 390 μl concentrated 97% sulfuric acid.

The weighed substance is added to 5 ml of 5% citric acid in a sealable vessel and dissolved by brief shaking. Add 20 μl (approx. 1 drop) of this solution to the first detection solution and compare the resulting color in the lower colored phase using the available reference dye. Alternatively, as in the other embodiments, the color can be evaluated by means of a spectrophotometer or APP and a calibration line can be used to draw a direct conclusion about the concentration present. Unwanted fillers that could influence the coloration are present in the clear uncolored phase and thus do not interfere with the detection of MDMA, so that a clear purple coloration is seen in the test and the concentration of the active ingredient can be determined by this coloration.

In order to detect unwanted extenders in the MDMA tablet, a further drop of 100 μl of the already weighed and dissolved substance is added to the second detection solution for “negative staining”, which contains 1 g sodium nitroprusside and 2 ml acetone in 50 ml distilled water. After mixing, this entire liquid is added to a glass vial containing 300 μl chloroform and 200 μl of a 2% sodium carbonate solution. In this coloration, MDMA is only very slightly colored pink, whereas extenders, such as amphetamine, have an intense coloration. This results in a strong orange coloration, which indicates the presence of amphetamine (colloquially known as speed).

The strength of this coloration can also be used to determine how much extender is present in this sample, which for the first time provides the user with “vital” information about the content of his sample. The color can now be used to identify the extender and the intensity of the resulting coloration can also be used to determine the concentration of this extender.

With the available information, dangerous overdoses can be prevented with undesirable mixed samples, which, for example, can be significantly more potent than MDMA and are added by default to illegal black-market drugs without the knowledge of the subsequent users.

This application example illustrates the first possibility of not only detecting substances that are stretched or mixed in some form by colorimetry, but also determining their concentration. The method can remove unwanted fillers, such as sugar and starch, and simultaneously test for different active ingredients and their concentrations.

### Design Example 7

Quantitative detection of possibly stretched indole alkaloids such as DMT, psilocybin, or other substances present in powder form (DMT is commonly smoked by consumers in powder form and extracted for this purpose from, for example, leaves of the plant Psychotria viridis). For the detection of various indole alkaloids, 20 mg of the powdered substance present is weighed out and dissolved in a vessel with 5 ml of a 5% aqueous citric acid solution by shaking. Even fruiting bodies of Psilocybe fungi can be used, but in the following step the biomass must be removed again by means of e.g. a sterile filter syringe before the solution can be added to the detection solution. Take 100 μl of the acidic extraction solution and add it to the detection solution in a second vessel. 50 ml of this detection solution contains 96% concentrated sulfuric acid with 0.1 g para-dimethylamino benzaldehyde and 0.1 g iron (III) chloride hexahydrate.

For the detection, 200 μl of this solution similar detection solution is mixed with 300 μl chloroform in a vessel and 100 μl of the acidic solution with the dissolved indole alkaloid is added.

The mixture is shaken several times and two phases are seen, one of which is clear and the other colored depending on the active ingredient contained. Psilocybin appears as a brown coloration and DMT as a yellowish coloration. It is also possible to identify completely different active ingredients, such as synthetic variants, which are immediately noticeable due to a change in coloration.

Possible extenders and fillers, such as in this case the indole alkaloids extracted from natural substances, are usually still present as purification residues, which collect in the clear uncolored chloroform layer and thus do not influence the coloration in the aqueous acid phase.

Also, common extenders that are added to the extracts for profit, such as various sugars, are also removed, and thus cannot influence or mask the coloration of the actual active ingredient and allow the actual analysis of the present active ingredient concentration.

### Design Example 8

Quantitative detection of tropane alkaloids, e.g. cocaine in powder form.

For the detection of tropane alkaloids, such as cocaine, 20 mg of the powdered substance is weighed out and dissolved in a vessel with 5 ml distilled water by shaking.

From this solution 100 μl are taken and added to the improved detection solution in a second vessel. 50 ml of this detection solution contains 1:1 5 g potassium nitrite in concentrated 97% sulfuric acid and 99% chloroform.

500 μl of the detection solution are present in a vessel to which the 100 μl of cocaine solution taken up is added. Again, two phases are formed, one containing cocaine and one containing possible fillers. The active ingredient content of cocaine can be read by the intensity of the yellow coloration formed in the stained lower phase (Through the possibilities for evaluation already described). A second detection solution is the use of cobalt(II)thiocyanate. The weighed substance is dissolved in distilled water, which turns blue in the presence of cocaine due to the cobalt(II)thiocyanate it contains. If 100 μl of the solution is now added to a second vessel containing 1:1 10% hydrochloric acid aqueous solution and chloroform, then a pink coloration is produced with a blue phase in the upper part of this. Since cocaine is actually almost always in an extended form, purification of the unwanted fillers is necessary to quantify the cocaine concentration. If extenders such as levamisole have been added to the cocaine, they are immediately noticeable in the first staining method by the orange coloration of the stained phase and can also be quantified by the intensity of the coloration using the evaluation methods described.

### Design Example 9

Quantitative detection of opiates, such as heroin, morphine and lethal extenders such as fentanyl.

For the detection of opiates such as heroin, morphine or fentanyl, 20 mg of the substance is weighed out and dissolved in a vessel

containing 5 ml distilled water by shaking. From this solution 100 μl are taken and added to the detection solution in a second vessel. 50 ml of this detection solution contains 4:1:5 (62% HNO₃:H₂O:CHCl₃).

From the detection solution, 500 μl are present in a vessel to which 100 μl of the absorbed opiate solution is added. Again, two phases are formed, one containing the stained active substance and a clear phase containing possible fillers.

The active ingredient content of heroin, for example can be read off from the intensity of the yellow coloration produced in the stained lower phase (with the aid of the evaluation options already described).

If the sample contains morphine, the test turns reddish. However, since heroin contains other extenders that resemble the yellowish color of heroin, a second test can be performed. A current example is the extender fentanyl, a synthetic opiate that is lethal at a dose of just a few milligrams.

For this purpose, the heroin solution already prepared can be added to a second detection solution. For this, 400 μl of chloroform is added to a 1.5 ml glass vessel with a screw cap. To this is added 10 μl formaldehyde and 390 μl concentrated 96% sulfuric acid.

From this opiate solution 100 μl are added to the second detection solution and the resulting color of the lower colored phase is compared using the available reference dye. Fentanyl can be immediately distinguished by an orange coloration from the light yellow of heroin or the red coloration from morphine.

Since heroin is actually almost always present in an extended form, purification of the unwanted fillers is necessary for quantification of the heroin. If extenders, such as fentanyl, have been added to the heroin, they will show up in a second staining, and can also be quantified by the intensity of the staining using the evaluation methods described.

By using this detection method, many deaths from the unintentional use of fentanyl in heroin could be avoided.

All features shown in the description, the embodiment examples and the following claims can be essential to the invention both individually and in any combination with one another.

### Abbreviations

tryptamine 2-(Indol-3-yl)ethylamine

NMT N-methyltryptamine

5-MeO-NMT 5-methoxy-N-methyltryptamine

dimethyltryptamine N,N-dimethyltryptamine

5-MeO-DMT 5-methoxy-N,N-dimethyltryptamine

5-bromo-DMT 5-bromo-N,N-dimethyltryptamine

bufotenine 5-hydroxy-N,N-dimethyltryptamine

N-methylserotonine 5-hydroxy-N-methyltryptamine

serotonin 5-hydroxytryptamine

psilocin 4-hydroxy-N,N-dimethyltryptamine

psilocybin 4-phosphoryloxy-N,N-dimethyltryptamine

baeocystin 4-phosphoryloxy-N-methyltryptamine

norbaeocystin 4-phosphoryloxytryptamine

melatonin 5-methoxy-N-acetyltryptamine

tryptophane α-carboxy-tryptamine

5-MeO-AMT 5-methoxy-a-methyltryptamine

AET α-ethyltryptamine

NET N-ethyltryptamine

DET N,N-diethyltryptamine

DiPT N,N-diisopropyltryptamine

DPT N,N-dipropyltryptamine

DBT N,N-dibutyltryptamine

4-HO-MET 4-hydroxy-N-methyl-N-ethyltryptamine

4-HO-DET 4-hydroxy-N,N-diethyltryptamine

4-PO-DET 4-phosphoryloxy-N,N-diethyltryptamine

4-HO-DIPT 4-hydroxy-N,N-diisopropyltryptamine

4-HO-MiPT 4-hydroxy-N-isopropyl-N-methyltryptamine

5-MeO-DALT 5-methoxy-N,N-diallyltryptamine

5-MeO-DiPT 5-methoxy-N,N-diisopropyltryptamine

sumatriptan 5-methylaminosulfonyl-N,N-dimethyltryptamin

methamphetamine N-methylamphetamine

oxilofrine 4-hydroxyephedrine

PMA 4-methoxyamphetamine

PMMA, Methyl-MA 2C-4-methoxy-N-methylamphetamine

2C-B 2,5-dimethoxy-4-bromophenethylamine

2C-C 2,5-dimethoxy-4-chlorphenethylamine

2C-D 2,5-dimethoxy-4-methylphenylethylamine

2C-E 2,5-dimethoxy-4-ethylphenylethylamine

2C-P 2,5-dimethoxy-4-propylphenethylamine

2C-I 2,5-dimethoxy-4-iodphenethylamine

2C-F 2,5-dimethoxy-4-fluorphenethylaminee

2C-N 2,5-dimethoxy-4-nitrophenethylamine

2C-T-2 2,5-dimethoxy-4-ethylthiophenylethylamine

2C-T-4 2,5-dimethoxy-4-(iso)-propylthiophenylethylamine

2C-T-7 2,5-dimethoxy-4-(n)-propylthiophenylethylamine

2C-T-8 2,5-dimethoxy-4-cyclopropylmethylthiophenylethylamin

2C-T-9 2,5-dimethoxy-4-butylthiophenylethylamine

2C-T-21 2,5-dimethoxy-4-(2-fluorethylthio)phenylethylamine

2C-TFM 2,5-dimethoxy-4-trifluormethylphenethylamine

MDA 3,4-methylendioxyamphetamine

MDMA MDEA,MDE 3,4-methylendioxy-N-methylamphetamine

MMDA-3a 3,4-methylendioxy-N-ethylamphetamine

MMDA 2-methoxy-3,4-methylendioxyamphetamine

mescaline (M) 3-methoxy-4,5-methylendioxyamphetamine

IM 3,4,5-trimethoxyphenylethylamine

TMPEA 2,3,4-trimethoxyphenylethylamine

2,4,5-trimethoxyphenylethylamine

1. Method for determining the concentration of arylalkylamine, pyrrole, Indole and opiate-derivatives comprising two process steps in the form of an extraction step and a subsequent analysis step using at least one color reagent, characterized in that the analysis step at least one color reagent with at least one arylalkylamine, pyrrole, indole or opiate derivative causes a quantitative and linear color reaction which can be measured by colorimetric methods and is detected and evaluated.

2. Method according to claim 1, characterized in that the color reagent is 4-(N,N-dimethylamino)benzaldehyde, Marquis reagent, Mecke reagent, Zwikker reagent, Simon's reagent, Mandelin reagent, Liebermann reagent, Froehde reagent, Folins reagent, Dille-Koppanyi reagent, Scott reagent or Duquenois-Levine reagent.

3. Method according to claim 1 or 2, characterized in that the color reaction proceeds over an incubation time, the color reaction is detected visually and the color values are compared with reference values for evaluation.

4. Method according to claim 3, characterized in that the reference values are a calibrated calibration solution, a color scale or a collection of individual measured values.

5. Method according to claim 1, 2 or 3, characterized in that during the incubation time the temperature is increased compared to the ambient temperature.

6. Use of the method according to one or more of claims 1 to 5 for rapid determination of the content of pyrrole, phenylethylamine or indole derivatives relevant as active ingredients from biological materials or in synthetic products.

7. Test kit for a rapid concentration determination of psilocybin, psilocin, LSD, DMT and wide indole derivatives, primary and secondary aromatic amines, opiates and substances similar to these active substances by means of the method according to one or more of claims 1 to 5 comprising:
a closed vessel, bag or other cavity in the form of a single or multiple test kit containing the required extraction solution as well as the required color reagent and other substances or solutions in a separate ampoule or other cavities, in which test kit a concentration determination is carried out by the addition of the test substance, the release of the color reagent and the subsequent incubation, and
instructions for extraction and subsequent concentration determination.

8. Test kit for a rapid concentration determination of psilocybin, psilocin, LSD, DMT and further indole derivatives, primary and secondary aromatic amines, opiates and substances similar to these active substances by means of the method according to one or more of claims 1 to 5 comprising:
a plurality of test strips containing color reagent, wherein a concentration determination is made on the test strips by the addition of the test substance, reaction with the color reagent and subsequent incubation, and
instructions for extraction and subsequent concentration determination.

9. Use of a method according to one or more of claims 1 to 5 and/or a test kit according to claim 7 or 8 for the rapid determination of the content of phenylethylamines, indole alkaloids, tropane alkaloids, opiates relevant as active substance, in which an analysis solution is added to an extraction solution for the analysis step, these solutions are mixed and subsequently two phases are formed, wherein at least
one color reagent is used for the analysis solution and the active substance in the colored phase is subjected to a concentration determination.
